(19) United States
(12) Patent Application Publication
(10) Pub. No.: US 2010/0322928 A1
(43) Pub. Date: Dec. 23, 2010

(54) METHODS OF DIAGNOSING, MONITORING TREATMENT AND TREATING SYSTEMIC LUPUS ERYTHEMATOSUS (SLE)

(75) Inventor: Edna Mozes, Rechovot (IL)

Correspondence Address: MARTIN D. MOYNIHAN d/b/a PRTSI, INC., P.O. BOX 16446, ARLINGTON, VA, 22215, US

(73) Assignee: Yeda Research and Development Co., Ltd., Rehovot (IL)

(21) Appl. No.: 12/448,937

(22) PCT Filed: Jan. 17, 2008
(86) PCT No.: PCT/IL08/00078

§ 371 Date: Jul. 16, 2009

Related U.S. Application Data
(60) Provisional application No. 60/881,202, filed on Jan. 19, 2007.

(51) Int. Cl. A61K 39/395; A61K 31/573; A61K 31/5377; A61K 38/16; A61K 31/713; A61P 37/02; A61P 29/00; C12Q 1/68; C40B 30/04

(52) U.S. Cl. 424/134.1; 514/179; 514/233.5; 514/16.6; 514/44 A; 424/133.1; 424/158.1; 514/21.2; 435/6; 506/9

(57) ABSTRACT

A method of treating systemic lupus erythematosus (SLE) in a subject are provided. The method comprise altering in cells of the subject activity and/or expression of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5R-alpha, A130040M12Rik, Gpr132, Cd8b1, Dhx9, Cyp11a1, Lmo7, Rnf184, Pstpip2, Hdgfrp3, Ass1 and Zbtb20, thereby treating SLE. Also provided are methods of diagnosing SLE and monitoring treatment of SLE.

### FIELD AND BACKGROUND OF THE INVENTION

The present invention, in some embodiments thereof, relates to methods of diagnosing, monitoring treatment and treating systemic lupus erythematosus (SLE).

Systemic lupus erythematosus (SLE) is an autoimmune disease characterized by the increased production of antibodies against several self-antigens and by defective T cell-mediated responses. The latter are associated with various clinical manifestations that involve multiple organs and tissues, including immune-complex depositions in the kidneys (1). A synthetic peptide (hCDR1; edratide) (2) based on the sequence of the complementarity-determining region (CDR) 1 of a human monoclonal anti-DNA antibody that bears the common idiotype 16/6Id (3,4), was shown to be capable of preventing an SLE-like disease or treating an already established disease (5). Beneficial effects of the peptide are manifested in the reduction of autoantibodies and the down-regulation of clinical symptoms including kidney damage (5). Studies aimed at elucidating the mechanisms that underlie the beneficial effects of edratide demonstrated that treatment of SLE-afflicted mice with edratide also resulted in reduced secretion and expression of “pathogenic” cytokines (i.e. IFNγ, IL-1β, TNFα, and IL-10), whereas the immunosuppressive cytokine TGFβ was up-regulated (5). Thus, the significant ameliorating effects of edratide are evidently manifested, at least in part, via immunomodulation of the cytokine profile (5-9).

U.S. Pat. No. 6,613,536 to Mozes, et al. discloses peptides based on the CDRs of mouse monoclonal antibodies (mAb) that are capable of inhibiting proliferative responses of T lymphocytes in SLE. Also, PCT Application WO 02/067848 discloses synthetic hCDR1 that can be used to immunomodulate SLE associated responses such as matrix metalloproteinase (MMP)-3, MMP-9, IL-2, IFNγ, and TGFβ. These enzymes and cytokines were either up-regulated or down-regulated because of SLE, and administration of edratide reversed these responses. However, there is no disclosure that genes were actually found to be up- or down-regulated. Treatment with edratide also reduced kidney disease in (NZB×NZW)F1 mice, a symptom associated with SLE.

The multiple clinical phenotypes of SLE are influenced by numerous genes. To date, more than 30 chromosomal regions containing genes affecting susceptibility or resistance to lupus have been identified in mouse models of SLE. Several of the susceptibility loci map to similar locations across various strains, notably in specific regions of chromosomes 1, 4, 7, and 17 (reviewed in 10). Susceptibility genes involved in a mouse model of induced SLE were found to map to chromosome 6, 7 and 14 (11). A number of studies have documented the contribution of major histocompatibility complex (MHC) (12,13) and non-MHC loci, such as CD22, PD-1, FcγRIIB and cytotoxic T-lymphocyte antigen 4, to lupus susceptibility (reviewed in 14). It thus seems that genes in multiple pathways participate in specific aspects of the disease. In humans, predisposition to SLE was shown to be influenced by the HLA region, complement components, and low-affinity receptors for IgG (reviewed in 10).

In attempting to unravel the complexity of SLE, a number of groups have employed microarray technology, a powerful tool for investigating differences in gene expression profiles in several diseases and their animal models. To characterize the complexity of immune dysregulation in lupus, some authors have used complementary DNA (cDNA)-arrays to study peripheral blood mononuclear cells (PBMCs) from lupus patients. Rus, et al. (15), using cytokine array membranes to compare gene expression patterns of PBMCs from SLE patients and healthy controls, identified 20 genes that differ significantly between patients and controls, and belong to a variety of families including the IL-1 family, TNF/death receptors, and IL-8 and its receptors. The same group subsequently described 29 additional genes that differentiate patients with active disease from those with inactive disease (16), and belong to various families including adhesion molecules, proteases, the TNF superfamily, and neurotrophic factors. Maas, et al. (17) reported differences in expression levels of genes encoding proteins that participate in apoptosis, cell-cycle progression, cell differentiation, and migration. Utilizing CD4+cells from lupus patients, other authors (18-20) have identified genes related to cellular development, Ras pathway, CD70 (19), cyclooxygenase-2 (COX-2) (20) and others. Also described were SLE-specific signature genes participating in DNA damage/repair pathways that result in production of nuclear autoantibodies (21).

In the only published study of (NZB×NZW)F1 mice, widely used as a model of SLE, the gene profile of nephritic (NZB×NZW)F1 kidneys was compared with those of non-diseased NZW controls (22). The most highly up-regulated gene (EDV, 5.5-fold) in the kidneys (but not in the spleens) of diseased mice corresponded to an endogenous retrovirus related to the Duplan strain (EDV, L08395).

U.S. Patent Application Number 20030148298 teaches methods for diagnosis and prognosis of Systemic lupus erythematosus by identifying differentially expressed genes. Moreover, the application is also directed to methods that can be used to screen test compounds and therapies for the ability to inhibit systemic lupus erythematosus. Additionally, methods and molecule targets (genes and their products) for therapeutic intervention in systemic lupus erythematosus.

The contents of all of the above documents are incorporated by reference as if fully set forth herein.

### SUMMARY OF THE INVENTION

According to an aspect of some embodiments of the present invention there is provided a method of treating systemic lupus erythematosus (SLE) in a subject, the method comprising upregulating in cells of the subject activity and/or expression of at least one gene selected from the group consisting of Mpo, Ltf, Lcn, Camp, Ngp, Slfn, Ctsg, Thbs1, S100a8, 1190003K14Rik, Prtn3, S100a9, and Tfpi, thereby treating SLE.

According to an aspect of some embodiments of the present invention there is provided a method of treating systemic lupus erythematosus (SLE) in a subject, the method comprising downregulating in cells of the subject activity and/or expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20, thereby treating SLE.

According to an aspect of some embodiments of the present invention there is provided a method of treating systemic lupus erythematosus (SLE) in a subject, the method comprising: (a) determining a level of expression of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject, so as to identify an altered expression of the at least one gene relative to a normal control sample; and (b) administering an anti SLE therapy to the subject according to the level of expression of the at least one gene, thereby treating SLE in the subject.

According to some embodiments of the invention, the method further comprising repeating step (a) following step (b).

According to an aspect of some embodiments of the present invention there is provided a method of monitoring treatment against systemic lupus erythematosus (SLE) in a subject, the method comprising: (a) administering an anti SLE therapy to the subject; and (b) determining a level of expression of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject following the administering, thereby monitoring treatment against the SLE in the subject.

According to some embodiments of the invention, the method further comprising performing step (b) prior to step (a).

According to an aspect of some embodiments of the present invention there is provided a method of diagnosing systemic lupus erythematosus (SLE) in a subject in need thereof, the method comprising determining a level of expression of at least one gene selected from the group consisting of Mpo, Ltf, Lcn, Camp, Ngp, Slfn, Ctsg, Thbs1, S100a8, 1190003K14Rik, Prtn3, S100a9, and Tfpi in cells of the subject, wherein an expression lower than a predetermined threshold of the at least one gene is indicative of SLE in the subject.

According to an aspect of some embodiments of the present invention there is provided a method of diagnosing systemic lupus erythematosus (SLE) in a subject in need thereof, the method comprising determining a level of expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject, wherein an expression higher than a predetermined threshold of the at least one gene is indicative of SLE in the subject.

According to an aspect of some embodiments of the present invention there is provided a kit for diagnosing systemic lupus erythematosus (SLE) the kit comprising agents directed for specific detection of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20.

According to some embodiments of the invention, the anti SLE therapy is selected from the group consisting of corticosteroids, an anti malarial, an NTHE, a DMARD, CellCept (mycophenolate mofetil; MMF), Orencia® (abatacept; CTLA4-Ig), Riquent™ (abetimus sodium; LJP 394), Prestara™ (praserone), Edratide (TV-4710), Actemra® (tocilizumab; atlizumab), VX-702, TRX 1, IPP-201101, ABR-215757, sphingosine-1-phosphate-1 (S1P1) agonist, HuMax-Inflam™ (MDX 018), MEDI-545 (MDX-1103/1333), RhuDex®, Deoxyspergualin, ENBREL™ (Etanercept), anti-TNF antibody, anti-interferon-alpha antibody and an anti Neutrokine-alpha protein.

According to an aspect of some embodiments of the present invention there is provided a method of monitoring treatment in a subject having systemic lupus erythematosus (SLE) comprising: (a) dministering to the subject a peptide as set forth in SEQ ID NO:1 (Edratide™); (b) nalyzing expression of at least one gene which expression level is altered in SLE following the administering; and (c) dentifying the subject as a responder to treatment with the peptide of SEQ ID NO:1 if gene expression level have been restored to normal, thereby monitoring SLE treatment.

According to some embodiments of the invention, the gene is selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1and Zbtb20.

According to some embodiments of the invention, aid cells comprise peripheral blood lymphocytes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are heat diagrams of differentially expressed genes in SLE-afflicted mice treated with edratide or with vehicle only. FIG. 1A shows genes affected by SLE, up-regulation is indicated in red and down-regulation in green, white lines represent genes whose expression was unchanged by treatment with edratide, block (a) shows genes that were changed in vehicle-treated SLE-afflicted mice relative to young disease-free mice, and block (b) shows the effect of edratide on the genes presented. FIG. 1B shows genes that were specifically regulated by more than 2 fold by the disease and oppositely affected by edratide. Up-regulation is indicated in red and down-regulation in green. Block (c) shows genes that were up-regulated by more than 2-fold in vehicle-treated SLE-afflicted mice relative to young disease-free mice, and block (d) shows genes that were down-regulated by more than 2-fold in vehicle-treated SLE-afflicted mice relative to young disease-free mice. Gene symbols and titles are listed in Tables 2 and 3;

FIGS. 2A-B show real-time RT-PCR analysis. Shown are mean±SD values of three independent experiments, each carried out in triplicates. Results were normalized to β-actin expression and are presented relative to vehicle treated mice (100%). FIG. 2A shows genes which were up-regulated by the disease and down-regulated by treatment with edratide. FIG. 2B shows genes which were down-regulated by the disease and up-regulated by treatment with edratide; and

FIGS. 3A-D show treatment with edratide down-regulates expression of OX40L in the kidneys of SLE-afflicted mice. Kidneys from the different groups were stained for detection of OX40L expression. Representative kidney sections from each group are shown. Staining was detected in glomeruli (FIG. 3A) and within the interstitial kidney tissue (FIG. 3B) of diseased mice that were treated with the vehicle only. FIG. 3C shows that expression of OX40L was down-regulated in the kidneys of diseased mice after treatment with edratide. FIG. 3D shows that expression of OX40L could not be detected in young disease-free (NZB×NZW)F1 mice. Magnification: ×400.

### DESCRIPTION OF EMBODIMENTS OF THE INVENTION

The present invention, in some embodiments thereof, relates to methods and kits for diagnosing SLE, monitoring treatment of SLE and treating SLE.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While reducing some embodiments of the present invention to practice, the present inventor has uncovered that administration of a peptide as set forth in SEQ ID NO: 1 (also referred to herein as Edratide) to an animal model of SLE, alleviates SLE symptoms and restores gene expression profile to that of a symptom free control sample. It is thus suggested that this newly uncovered set of genes is associated with alleviation of SLE symptoms, substantiating its use in therapeutics and diagnostics of SLE.

Specifically, as shown in the Examples section which follows, NZB×NZWF 1 mice (SLE-mouse model) treated with Edratide or control vehicle (Captisol) exhibited major differences in gene expression. As can be seen from Tables 2-3 below, treatment with Edratide restored gene expression pattern to that of 8 weeks-old NZB×NZWF1 mice which do not present with clinical signs of SLE. These results strongly suggest that gene expression analysis can be used for monitoring treatment with anti SLE therapy in general and Edratide in particular.

Thus, according to once aspect of the present invention there is provided a method of monitoring treatment of a subject having systemic lupus erythematosus (SLE). The method comprising administering to the an anti SLE therapy; analyzing expression of at least one gene which expression level is altered in SLE following said administering; and identifying the subject as a responder to treatment with the anti SLE therapy if gene expression levels have been restored to normal, thereby monitoring SLE treatment.

The phrase “Systemic lupus erythematosus” is interchangeably used herein with SLE and lupus and refers to all manifestations of the disease as known in the art (including remissions and flares).

As used herein the phrase “subject in need thereof” refers to a human subject of any sex or age that is suspected of having SLE, diagnosed with, or predisposed to SLE.

As used herein the terms “treatment” and “treating” refer to preventing, curing, reversing, attenuating, alleviating, minimizing, suppressing or halting the deleterious effects of the disease (SLE).

As used herein the phrase “monitoring treatment” refers to determining therapeutic efficacy of anti SLE therapy. Determination can be qualitative or quantitative and can be further improved using standard methods of diagnosing SLE e.g., ACR classification criteria.

As used herein the phrase “anti SLE therapy” refers to any physical (e.g., UVA-1 phototherapy), chemical, genetic, surgical and life style treatment (e.g., avoiding sunlight and weight loss) which is known in the art for the treatment of SLE or its symptomatic manifestations.

The following provides a non-limiting description of anti SLE therapies which can be used in accordance with the teachings of the present invention.

Nonsteroidal Anti-Inflammatory Drugs (NSAIDs)—NSAIDs are often used to reduce pain and inflammation in patients who have mild systemic lupus erythematosus (SLE). Examples of NSAIDs which can be used in accordance with the present teachings include aspirin, Motrin, Orudis, and Anaprox. DMARDs are often used to help control the disease. Typically methotrexate, a disease-modifying antirheumatic drug is used.

Antimalarials—Antimalarials are another type of drug commonly used to treat lupus. These drugs were originally used to treat malaria, but doctors have found that they also are useful for lupus. A common antimalarial used to treat lupus is hydroxychloroquine (Plaquenil).

Corticosteroids—Corticosteroids are very powerful drugs that reduce inflammation in various tissues of the body. These drugs are used to treat many of the symptoms of lupus that result from inflammation. Prednisone is a corticosteroid that is often used to treat lupus. Other corticosteroids which may be used in accordance with the present teachings include, but are not limited to, prednisolone, hydrocortisone, methylprednisolone or dexamethasone.

Immunosuppressives—Azathioprine is a drug that acts to suppress the work of the immune system. It is used mainly in organ transplantation to prevent the body from rejecting the new organ. The drug is also used in patients with lupus who have damage to their kidneys or other organs, muscle inflammation, or advanced arthritis. Azathioprine helps to reduce symptoms and damage to the affected organs. It can also help achieve a remission of the disease. Mycophenylate mofetil is another new alternative immunosuppressive drug. The combination of a corticosteroid and an immunosuppressive drug is most often used for severe kidney disease or nervous system disease and for vasculitis. Cyclophosphamide is a drug used to treat a number of cancers, and it is used to treat patients with lupus when major organs, such as the kidneys, are affected. It is also used to treat severe inflammation that has not responded to corticosteroids. In lupus, the immune system is too active. Cyclophosphamide slows down the immune system so that disease activity can be reduced.

In a specific embodiment, the methods of the present invention may be practiced with one or more of the following drugs: CellCept (mycophenolate mofetil; MMF), Orencia® (abatacept; CTLA4-Ig), Riquent™ (abetimus sodium; LJP 394), Prestara™ (praserone), Edratide (TV4710), Actemra® (tocilizumab; atlizumab), VX-702, TRX 1, IPP-201101, ABR-215757, sphingosine-1-phosphate-1 (S1P1) agonist, HuMax-Inflam™ (MDX 018), MEDI-545 (MDX-1103/1333), RhuDex® Deoxyspergualin, ENBREL™ (Etanercept), rapamycin, anti-TNF antibody, anti-interferon-alpha antibody.

Administration route, dosage and formulation will off course depend on the selected drug and severity of the symptoms (quality and frequency).

Gene expression is determined in immunocytes of the subject (e.g., cells of the immune system such PBMC/PBL). Preferably, expression levels in the analyzed sample are compared with expression levels of the same gene from a control individual. It is preferable that the control sample comes from a subject of the same species, age and from the same sub-tissue. Alternatively, control data may be taken from databases and literature.

Conceivably analyzing expression levels and administering steps may be repeated a number of times during the course of a treatment. For instance gene expression levels may be analyzed one week following treatment. If the levels are still higher or lower than those compared with a control healthy sample, dosage may be increased.

As mentioned determining the need for SLE treatment (i.e., administration of anti SLE therapy) is by analyzing gene expression of the genes listed herein (see Tables 2-3 hereinbelow). In doing so, additional information may be gleaned pertaining to the determination of treatment regimen, treatment course and/or to the measurement of the severity of the disease.

Thus, embodiments of the present invention further provide a method of diagnosing systemic lupus erythematosus (SLE) in a subject in need thereof, the method comprising determining a level of expression of at least one gene selected from the group consisting of Mpo, Ltf, Lcn, Camp, Ngp, Slfn, Ctsg, Thbs 1, S100a8, 1190003K14Rik, Prtn3, S100a9, and Tfpi in cells of the subject, wherein an expression lower than a predetermined threshold of the at least one gene is indicative of SLE in the subject.

Alternatively or additionally diagnosing of SLE can be effected by determining a level of expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject, wherein an expression higher than a predetermined threshold of the at least one gene is indicative of SLE in the subject.

As used herein the term “diagnosing” refers to determining the presence of a disease, classifying a disease, determining a severity of the disease (grade or stage), monitoring disease progression, forecasting an outcome of the disease and/or prospects of recovery.

Methods of determining gene expression at the protein or mRNA level are described infra. The description is not meant to be limiting and other methods are well known and may be used by those of skill in the art.

Methods of Detecting the Expression Level of RNA

The expression level of the RNA in the cells of the present invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (ie., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. The oligonucleotide array of some embodiments of the present invention comprises less than 500 oligonucleotide probes. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in the cells of the cultures of the present invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Protein arrays which comprise antibodies for the detection of presence/level of protein expression products of those genes listed in Tables 2-3 below are also contemplated by the present teachings. Methods of fabricating protein arrays are well known in the art (see e.g., Cahill (2000) Trends in Biotechnology 18:47-51).

Specific examples of genes which expression may be screened in accordance with some embodiments of the present invention are listed in Tables 2-3 below.

Other genes which expression may be analyzed in association with Edratide treatment are listed in U.S. Patent Application Number 20030148298, the teachings of which are hereby incorporated by reference.

Restoration of gene expression following treatment with Edratide and alleviation of clinical symptoms, suggests that the genes listed in Tables 2-3 below, can be used as therapeutic tools/targets for treating SLE.

Thus, according to another aspect of the present invention there is provided a method of treating systemic lupus erythematosus (SLE) in a subject, the method comprising upregulating in cells of the subject activity and/or expression of at least one gene selected from the group consisting of Mpo, Ltf, Lcn, Camp, Ngp, Slfn, Ctsg, Thbs1, S100a8, 1190003K14Rik, Prtn3, S100a9, and Tfpi, thereby treating SLE.

According to yet another aspect of the present invention there is provided a method of treating systemic lupus erythematosus (SLE) in a subject, the method comprising downregulating in cells of the subject activity and/or expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20, thereby treating SLE.

Methods of upregulating and down-regulating gene expression are outlined hereinbelow. The description is not meant to be limiting and other methods are well known and may be used by those of skill in the art.

Upregulation

Upregulation of gene expression can be effected at the genomic level (i.e., activation of transcription via promoters, enhancers, regulatory elements), at the transcript level (i.e., correct splicing, polyadenylation, activation of translation) or at the protein level (i.e., post-translational modifications, interaction with substrates and the like).

Following is a list of agents capable of upregulating the expression level and/or activity of the genes of interest (listed in Table 3, below).

An agent capable of upregulating expression of a gene may be an exogenous polynucleotide sequence designed and constructed to express at least a functional portion of the gene. Accordingly, the exogenous polynucleotide sequence may be a DNA or RNA sequence encoding the gene of interest.

The phrase “functional portion” as used herein refers to part of the protein (i.e., a polypeptide) which exhibits functional properties of the enzyme such as binding to a substrate.

To express exogenous genes in mammalian cells, a polynucleotide sequence encoding the polypeptide product is preferably ligated into a nucleic acid construct suitable for mammalian cell expression. Such a nucleic acid construct includes a promoter sequence for directing transcription of the polynucleotide sequence in the cell in a constitutive or inducible manner.

It will be appreciated that the nucleic acid construct of the present invention can also utilize homologous sequences which exhibit the desired activity. Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the genes listed in Table 3 below, as determined using the BestFit software of the Wisconsin sequence analysis package, utilizing the Smith and Waterman algorithm, where gap weight equals 50, length weight equals 3, average match equals 10 and average mismatch equals −9.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M, et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types.

Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p 205. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo gene expression since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

It will be appreciated that upregulation of gene expression can be also effected by administration of genetically modified cells into the individual (i.e., ex-vivo gene therapy).

Such cells can be stem cells which have long been suggested for the treatment of SLE.

Administration of the cells of the present invention can be effected using any suitable route such as intravenous, intra peritoneal, intra kidney, intra gastrointestinal track, subcutaneous, transcutaneous, intramuscular, intracutaneous, intrathecal, epidural and rectal. According to presently preferred embodiments, the cells of the present invention are introduced to the individual using intravenous, intra kidney, intra gastrointestinal track and/or intra peritoneal administrations.

Cells of the present invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Downregulating

Downregulation of gene expression e.g., genes which are listed in Table 2 below can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of downregulating expression level and/or activity of the genes listed in Table 2 below.

One example, of an agent capable of downregulating a protein product is an antibody or antibody fragment capabale of specifically binding the protein product. Preferably, the antibody specifically binds at least one epitope. As used herein, the term “epitope” refers to any antigenic determinant on an antigen to which the paratope of an antibody binds.

Antibodies are typically used for extracellular epitopes (e.g., anchored to the cell surface e.g.,CD8 antigen beta chain 1 and TNF ligand)

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Another agent capable of downregulating gene expression is a small interfering RNA (siRNA) molecule. RNA interference is a two step process. The first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 by duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Other agents capable of downregulating gene expression include, but are not limited to DNAzymes molecule capable of specifically cleaving an mRNA transcript or DNA sequence of the gene of interest; antisense polynucleotides capable of specifically hybridizing with an mRNA transcript encoding the gene of interest; ribozyme molecules capable of specifically cleaving an mRNA transcript encoding the gene of interest; and triplex forming oligonucleotides (TFOs).

Diagnostic and therapeutic agents contemplated by the present teachings may be included in a diagnostic therapeutic kit/article of manufacture preferably along with appropriate instructions for use and labels indicating FDA approval for use in diagnosing SLE, for monitoring efficacy of SLE therapy and/or for treating SLE.

Such a diagnositc kit can include, for example, at least one container including at least one of the above described diagnostic agents and an imaging reagent packed in another container (e.g., enzymes, secondary antibodies, buffers, chromogenic substrates, fluorogenic material). The kit may also include appropriate buffers and preservatives for improving the shelf-life of the kit.

It is expected that during the life of a patent maturing from this application many relevant reagents will be developed and the scope of the terms used herein is intended to include all such new technologies a priori.

As used herein the term “about” refers to ±10%. The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases to “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Mice. Female (NZB×NZW)F1 mice were obtained from The Jackson Laboratory (Bar Harbor, Me.). The study was approved by the Animal Care and Use Committee of the Weizmann Institute of Science.

Synthetic peptides. The peptide used in these Examples was edratide, which is based on the CDR1 of the human monoclonal anti-DNA antibody bearing the common idiotype designated 16/6Id (4). This peptide (GYYWSWIRQPPGKGEEWIG-SEQ ID NO 1) was synthesized (solid phase synthesis by Fmoc chemistry) by PolyPeptide laboratories (Torrance, Calif.). Edratide (hCDR1) is currently under clinical development for treating human SLE by Teva Pharmaceutical Industries (Israel).

Vehicle. The vehicle used was Captisol® (sulfobutylether beta cyclodextrin) a solvent designed by CyDex (Lenexa, Kans.), to enhance the solubility and stability of drugs.

Treatment of (NZB×NZW)F1 mice with edratide. Female mice aged 6-7 months, with established SLE, were divided into groups (8-10 mice per group) and injected subcutaneously (s.c.) with edratide (25-50 μg) or vehicle only (Captisol®) once a week for 10 weeks. Proteinuria and anti-double stranded DNA (dsDNA) autoantibodies were monitored throughout this period.

Proteinuria. Proteinuria was measured by a standard semi-quantitative test, using an Albustix kit (Bayer Diagnostics, Newbury, UK).

Immunohistology. For detection of immune-complex deposits, frozen kidney sections (6 μm) were air-dried and fixed in acetone. Sections were incubated with FITC-conjugated goat anti-mouse IgG (gamma-chain specific; Jackson ImmunoResearch Laboratories, Avondale, Pa.) for 30 minutes and were extensively washed with PBS. The intensity of immune-complex deposits was graded on a scale of 1-3: 0, no deposits; 1, low-intensity deposits; 2, moderate-intensity deposits; 3, high-intensity deposits.

Immunohistochemistry of kidney sections. Frozen cryostat sections (6 μm) were air-dried and fixed in acetone. For detection of OX40 ligand (OX40L) expression, sections were incubated for 16 hours at room temperature with anti-OX40L monoclonal antibody (M−20; Santa Cruz Biotechnology, Santa Cruz, Calif.). FITC-conjugated mouse anti-goat IgG (Jackson ImmunoResearch Laboratories, West Grove, Pa.) was then added.

Enzyme-linked immunosorbent assay (ELISA) for the detection of dsDNA-specific antibodies. Anti-dsDNA-specific antibodies were determined using lambda phage dsDNA as previously described (5).

Preparation of RNA. Using the RNA/DNA/protein isolation reagent (TRI Reagent®; Molecular Research Center, Cincinnati, Ohio), total RNA was isolated from spleen cells that were pooled (n=2-4 mice) from each experimental group of (NZB×NZW)F1 mice.

Microarray and statistics. RNA samples were hybridized to the mouse genome AffymetrixGeneChip® 430A2 (Affymetrix, Santa Clara, Calif.) array. R packages were used from the Bioconductor project (23). Initially, probe signal summarization, normalization, and background subtraction were performed using Robust Multi-array Averaging (RMA) (24) in affy package with default parameters. Then, the statistical test for differentially expressed genes was performed using Linear Models for Microarray (LIMMA) package (25), which allows for a better variance estimation by calculating the moderated t-statistic using empirical Bayesian techniques. Finally, genes were selected that were statistically significant with P value <0.05 in each biological comparison of interest separately.

Real-time RT-PCR. Messenger RNA (mRNA) was analyzed by quantitative real-time RT-PCR using LightCycler (Roche Diagnostics, Mannheim, Germany). Total RNA was isolated from spleen cells pooled from each treatment group (n=2−4) of (NZB×NZW)F1 mice. To prepare cDNA, RNA was reverse-transcribed using Moloney murine leukemia virus (M-MLV) reverse transcriptase (Promega, Madison, Wis.). The resulting cDNA was subjected to real-time RT-PCR according to the manufacturer's instructions. Briefly, the reaction volume (20 μl) contained 3 mM MgCl₂, LightCycler hot start DNA SYBR Green I mix (Roche), specific primer pairs, and 5 μl of cDNA. Conditions for PCR were as follows: 10 minutes at 95° C. followed by 35-50 cycles of 15 seconds at 95° C., 15 seconds at 60° C., and 15 seconds at 72° C. Primer sequences (forward and reverse, respectively) were used as follows:

| β-actin: | |
|---|---|
| 5′-GTGACGTTGACATCCG-3′, | (SEQ ID NO: 2) |
| 5′-CAGTAACAGTCCGCCT-3′. | (SEQ ID NO: 3) |
| Nid1: | |
| 5′-AGTTCGGTTTGCATGG-3′, | (SEQ ID NO: 4) |
| 5′-GTAAGCAGGTCGAGGTG-3′. | (SEQ ID NO: 5) |
| Tnfsf4: | |
| 5′-CATGCTATTGTATGCCGAG-3′, | (SEQ ID NO: 6) |
| 5′-CGTCACCTATGGTCACT-3′. | (SEQ ID NO: 7) |
| Zbtb20: | |
| 5′-TCGAAATCCCGTCGGT-3′, | (SEQ ID NO: 8) |
| 5′-GCGGAGTAGATTCGGT-3′. | (SEQ ID NO: 9) |
| IL5R: | |
| 5′-ACCAGTTTAGCCAATTATGT-3′, | (SEQ ID NO: 10) |
| 5′-CCAGCAATCACCTCCA-3′. | (SEQ ID NO: 11) |
| S100a8: | |
| 5′CCGTCTGAACTGGAGAAG-3′, | (SEQ ID NO: 12) |
| 5′-CCAGAAGCTCTGCTACT-3′. | (SEQ ID NO: 13) |
| Tfpi: | |
| TTCGTGTACGGTGGCT-3′, | (SEQ ID NO: 14) |
| 5′-ACGATAATCCCGACGC-3′. | (SEQ ID NO: 15) |

β-actin levels were used for normalization in calculating the expression levels of all other genes.

### Results

Treatment with edratide ameliorates disease manifestations in (NZB×NZW)F1 mice with established SLE. SLE-afflicted female mice, aged 6-7 months, were treated with 10 weekly s.c. injections of edratide or vehicle (Captisol®) and followed for disease manifestations. One week after the treatment ended the mice were killed and their kidneys were evaluated for immune-complex deposits. The results of a representative experiment are presented in Table 1 below, which summarizes some clinical manifestations of the experimental mice.

TABLE 1
Effect of treatment with edratide on clinical manifestations of SLE*

| | Anti dsDNA antibodies^{†} | | | |
|---|---|---|---|---|
| | (OD) | | | |
| | Dilution | Dilution | Proteinuria^{††} | Intensity of |
| | 1:10 | 1:250 | (g/l) | ICD^{†††} |
| Vehicle | 3.10 ± 0.19 | 1.64 ± 0.48 | 10.75 ± 3.09 | 2.75 ± 0.46 |
| edratide | 1.46 ± 0.32 | 0.48 ± 0.21 | 1.92 ± 0.96 | 1.33 ± 0.36 |
| P value | 0.0009 | 0.0062 | 0.02 | 0.01 |

*Data are from 1 representative experiment of 3 performed. Mice (n = 8-10 per group) were injected s.c. Treatment was given once a week for 10 weeks.
^{†}Measured in sera from mice that were bled after termination of treatment.
^{††}Determined at the end of the experiment.
^{†††}ICD = Immune-complex deposits. Intensity of the deposits was graded on a scale of 1-3: 0, no immune-complex deposits; 1, low intensity; 2, moderate intensity; 3, high intensity.

It can be seen in Table 1 that mice treated with vehicle alone exhibited high levels of anti-dsDNA autoantibodies. In mice treated with edratide, however, these levels were significantly reduced. Treatment with edratide also ameliorated kidney disease, as indicated by a decrease both in proteinuria and in the intensity of immune-complex deposits in the kidneys relative to mice treated with vehicle alone. Proteinuria, anti-dsDNA autoantibodies, and immune-complex deposits could not be detected in young disease-free control mice.

Microarray analysis of immunocytes from SLE-afflicted mice. In an attempt to identify gene expression profiles characteristic of SLE and to gain an insight into the modified profile induced by edratide, DNA microarray technology was used on RNA preparations from splenocytes of SLE-afflicted (NZB×NZW)F1 mice treated with edratide or with vehicle only (as described above and in Table 1). As a control, RNA samples were prepared from the spleen cells of mice aged 2 months, an age at which the mice do not exhibit any of the clinical manifestations typical of SLE. Three independent in vivo experiments were successfully performed. The RNA obtained in each experiment was hybridized independently on the Affymetrix chips.

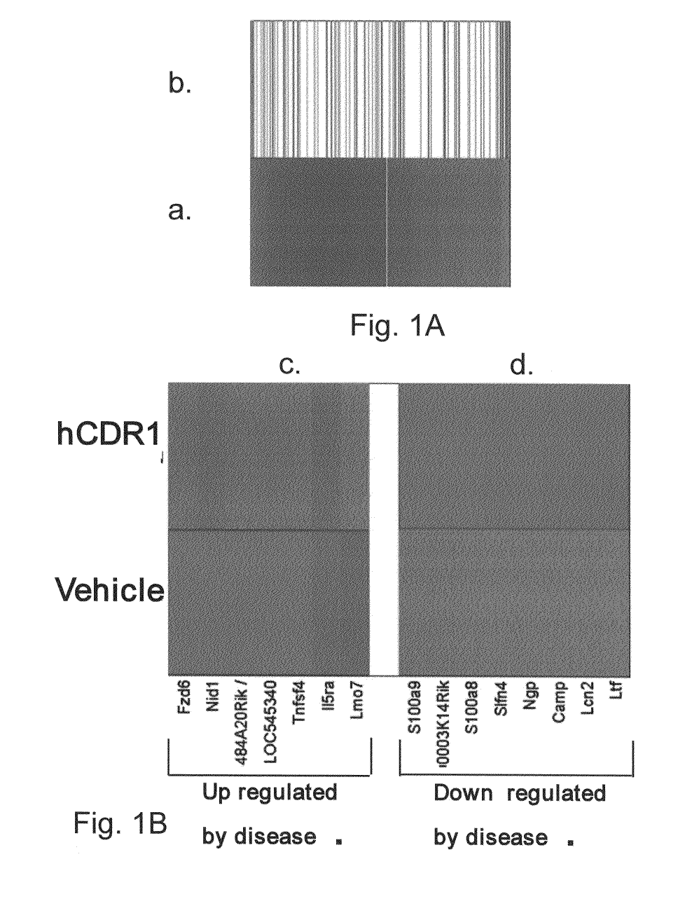

Of the ˜22,000 genes tested by the microarray experiment, the expression of 348 genes in the vehicle-treated SLE-afflicted mice differed significantly (P=0.05) from that of healthy young controls. The differently expressed genes are graphically represented in a heat diagram (FIG. 1A). In block (a), red lines represent the 183 genes that were up-regulated and green lines represent the 165 genes that were down-regulated in diseased mice relative to 2-month-old, disease-free (NZB×NZW)F1 mice.

To gain an insight into the modified gene expression profile involved in the ameliorating effect of edratide, 76 genes were focused on (22% of the 348 with altered expression in the vehicle-treated diseased mice) that were affected by treatment with edratide, and whose RNA expression was restored to levels similar to those observed in the disease-free controls. In block (b) of FIG. 1A these 76 genes are marked by red lines or green lines representing, respectively, genes that were up-regulated or down-regulated in edratide-treated mice relative to vehicle-treated diseased mice. White lines represent genes that were not affected by edratide treatment. The results in block (b) show that the effect of edratide on transcript level is reciprocal to the disease, and many genes (76 genes, ˜22%) are regulated as a result of edratide treatment and show the opposite trend to that seen in diseased mice.

The heat diagram in FIG. 1B shows the genes that were up- or down-regulated in SLE-afflicted mice by 2-fold or more relative to young mice, and were affected by edratide. Block (c) shows the genes that were up-regulated by the disease (red lines). Treatment with edratide down-regulated these genes, as indicated by the green lines. Block (d) shows genes that were down-regulated by the disease (green lines) and were up-regulated after treatment with edratide (red lines).

Of the 15 genes with an identified product that were up-regulated by 2-fold or more in SLE-afflicted mice, 9 (60%) were found to be significantly down-regulated after treatment with edratide. Table 2 below lists these genes and summarizes the magnitude of their changes.

TABLE 2
Genes that are up-regulated in SLE-afflicted mice*

| | | SLE | edratide | Cellular |
|---|---|---|---|---|
| Gene title | Gene symbol | (fold) | (fold) | location |
| Frizzled homolog 6 (Drosophila) | Fzd6 | 2.6 | −2.9 | Membrane |
| Nidogen 1 | Nid1 | 2.4 | −2.4 | ECM |
| RIKEN cDNA 5830484A20 | 5830484A20Rik | 2.4 | −3.0 | Nucleus |
| Similar to RIKEN cDNA 5830484A20 | LOC 545340 | 2.3 | −2.7 | Unknown |
| Tumor necrosis factor (ligand) | Tnfsf4 | 2.3 | −2.5 | Membrane |
| superfamily, member 4 | | | | |
| Proline-serine-threonine | Pstpip2 | 2.2 | −1.9^{†} | Cytoskeleton |
| phosphatase-interacting protein 2 | | | | |
| Polymeric immunoglobulin receptor | Pigr | 2.2 | −1.4^{†} | Membrane |
| RIKEN cDNA 2700022B06 gene | 2700022B06Rik | 2.1 | −1.4^{†} | Unknown |
| Interleukin 5 receptor, alpha | IL5Rα | 2.1 | −2.0 | Membrane |
| RIKEN cDNA A130040M12 | A130040M12Rik | 2.1 | 1.3^{†} | Unknown |
| G protein-coupled receptor 132 | Gpr132 | 2.1 | −1.1^{†} | Membrane |
| CD8 antigen, beta chain 1 | Cd8b1 | 2.1 | 2.0^{†} | Membrane |
| DEAH (Asp-Glu-Ala-His) box | Dhx9 | 2.0 | −1.0^{†} | Nucleus |
| polypeptide 9 | | | | |
| Cytochrome p450, family 11, | Cyp11a1 | 2.0 | −1.8^{†} | Mitochondrion |
| subfamily a, polypeptide 1 | | | | Membrane |
| LIM domain only 7 | Lmo7 | 2.0 | −2.8 | Unknown |
| Ring finger protein 184 | Rnf184 | 2.0 | 1.2^{†} | Unknown |
| Proline-serine-threonine | Pstpip2 | 2.0 | −1.5^{†} | Cytoskeleton |
| Phosphatase-interacting protein 2 | | | | |
| Unknown | Unknown | 1.9 | −1.7 | Unknown |
| Hepatoma-derived growth factor, | Hdgfrp3 | 1.9 | −1.7^{†} | Nucleus |
| related protein 3 | | | | |
| Zinc finger and BTB domain | Zbtb20 | 1.9 | −2.2 | Nucleus |
| containing 20 | | | | |
| Argininosuccinate synthetase 1 | Ass1 | 1.9 | −1.2^{†} | Mitochondrion |

*Results are presented as the fold difference between control mice and SLE-afflicted mice treated with vehicle only (designated “SLE”) and between edratide-treated mice (designated “hCDR1”) and SLE mice. Listed are genes that were up-regulated by 2 fold or more. A negative fold change represents a reduction in signal.
^{†}Not statistically significant. ECM = Extracellular matrix.

Of the 12 identified genes that were down-regulated by 2-fold or more in the diseased mice, 9 (75%) were up-regulated after edratide treatment (Table 3, below). Notably, of the genes for which no product has yet been identified, 5 were up-regulated and 1 was down-regulated in diseased mice.

TABLE 3
Genes that are down-regulated in SLE-afflicted mice*

| | | SLE | hCDR1 | Cellular |
|---|---|---|---|---|
| Gene title | Gene symbol | (fold) | (fold) | location |
| Myeloperoxidase | Mpo | −3.2 | 1.5^{†} | ES |
| Lactotransferrin | Ltf | −2.8 | 3.2 | ES |
| Lipocalin 2 | Lcn | −2.7 | 2.9 | ES |
| Cathelicidin | Camp | −2.6 | 2.8 | ES |
| antimicrobial peptide | | | | |
| Neutrophilic granule | Ngp | −2.5 | 2.5 | ES |
| protein | | | | |
| Schlafen 4 | Slfn | −2.4 | 2.7 | Unknown |
| Cathepsin G | Ctsg | −2.4 | 1.2^{†} | ES |
| Thrombospondin 1 | Thbs1 | −2.3 | 1.5^{†} | ES |
| S100 calcium binding | S100a8 | −2.3 | 2.4 | Unknown |
| protein A8 | | | | |
| (calgranulin A) | | | | |
| RIKEN cDNA | 1190003K14Rik | −2.2 | 2.4 | Unknown |
| 1190003K14 | | | | |
| Proteinase 3 | Prtn3 | −2.2 | 1.3^{†} | ES |
| S100 calcium binding | S100a9 | −2.2 | 2.4 | Unknown |
| protein A9 | | | | |
| (calgranulin B) | | | | |
| Tissue factor pathway | Tfpi | −2.0 | 2.2 | ES |
| Inhibitor | | | | |

*Results are presented as the fold difference between control mice and SLE-afflicted mice treated with vehicle only (designated “SLE”) and between edratide-treated mice (designated “hCDR1”) and SLE mice. Listed are genes that were down-regulated by 2-fold or more. A negative fold change represents a reduction in signal.
^{†}Non statistically significant. ES = extracellular space.

Seven of the 15 genes that were up-regulated by 2-fold or more in the diseased mice (Table 2) encode for membrane-associated proteins. Six of these gene products are located in the plasma membrane and one—cytochrome P450—in the mitochondrial membrane. Of the 12 genes that were down-regulated by 2-fold or more in the diseased mice (Table 3), the gene products of 9 are known to be extracellular proteins. Of the 10 gene products with known activity, 3 are enzymes and 2 are protease inhibitors. Moreover, of the 12 that were down-regulated by more than 2-fold, all but 2 (Schlafen 4 and thrombospondin) were identified as genes that encode proteins known to be associated with monocytes.

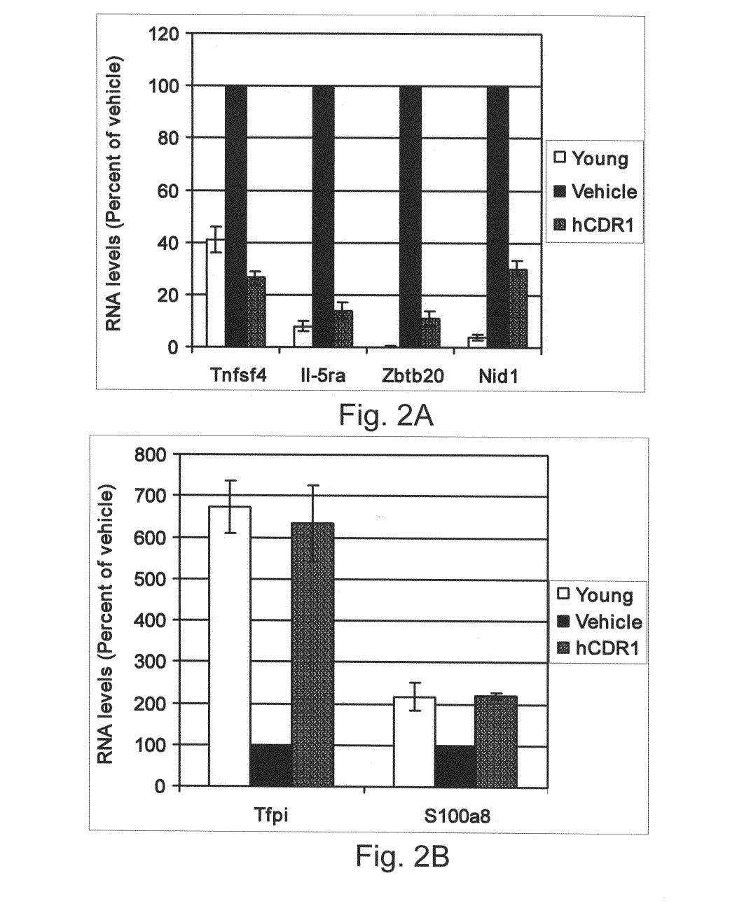

Real-time RT-PCR analysis. To confirm the microarray results, independent testing was carried out by real-time RT-PCR of the expression patterns of 6 representative genes that were significantly up-regulated (Tnfsf4, IL5Rα, Nid1, and Zbtb20) or down-regulated (S100a8 and Tfpi) in the SLE-afflicted mice and oppositely affected by edratide treatment. These genes were chosen because they reportedly meet at least one of the following three criteria: involvement in SLE, functioning in lymphocytes, and expression in the kidney. Results of the microarray analyses were confirmed by real-time RT-PCR (FIG. 2A), which showed marked increase of Tnfsf4, Il-5rα, Zbtb20, and Nid1transcripts (2.4 fold, 12.5 fold, 25 fold, and 417 fold, respectively) in diseased mice relative to the young, disease-free controls. Treatment with edratide reduced the expression of these genes to levels similar to those seen in the young mice. Likewise, transcripts of Tfpi and S100a8, which were down-regulated (6.7-fold and 2.2-fold, respectively) in vehicle-treated diseased mice, were up-regulated after edratide treatment to levels similar to those in the young controls (FIG. 2B).

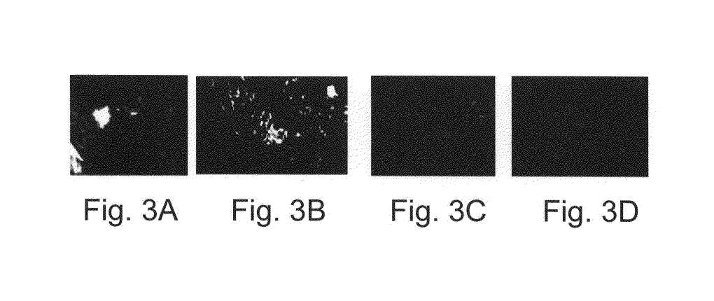

Immunostaining of kidneys of (NZB×NZW)F1 mice for OX40L expression. The results showed that transcripts of Tnfsf4, which encodes for OX40L, are differentially expressed in spleen-derived cells from SLE-afflicted mice and young controls, and that the mRNA was restored to approximately control levels after treatment with edratide. In view of the reported presence of OX40L (26) in kidney biopsies from all tested lupus patients with proliferative lupus nephritis, it was of interest to determine whether this ligand is also up-regulated in the kidneys of diseased (NZB×NZW)F1 mice, and whether—as shown for the transcripts in spleen cells—its expression is affected by treatment with edratide. Immunohistological analysis of kidney sections from 5 SLE-afflicted vehicle-treated or 6 edratide-treated mice and 3 disease-free young mice was performed. The results revealed intense OX40L immunostaining of the glomeruli and/or interstitial tissue in all 5 vehicle-treated mice, whereas OX40L expression in kidney sections of both the edratide-treated mice and the disease-free controls was very weak (in 2 mice and 1 mouse, respectively) or undetectable (in 4 and 2 mice, respectively). FIG. 3 shows representative results of immunostaining for OX40L in the glomeruli (A) and interstitial tissue (B) of a vehicle-treated mouse, as well as kidney sections from an edratide-treated mouse (C) and a young disease-free mouse (D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Document

1. Hahn B H, An overview of the pathogenesis of systemic lupus erythematosus, in: Dubis' Lupus Erythomatosus. D. J. Wallace, B. H. Hahn (Eds.), Williams and Wilkins, Philadelphia, Pa., 1993, p. 69-76.
2. Sthoeger Z M, Dayan, M, Tcherniack A, Green L., Toledo S., Segal R., Elkayam O, Mozes E. Modulation of autoreactive responses of peripheral blood lymphocytes of patients with systemic lupus erythematosus by peptides based on human and murine anti-DNA autoantibodies, Clin. Exp. Immunol. 2003; 131:385-92.
Mendlovic S., Brocke, S., Shoenfeld Y., Ben Bassat M., Meshorer A., Bakimer R., Mozes E. Induction of a systemic lupus erythematosus-like disease in mice by a common human anti-DNA idiotype, Proc. Natl. Acad. Sci. USA 1988; 85:2260-4.
4. Waisman A, Shoenfeld Y, Blank M, Ruiz P J, Mozes E. The pathogenic human monoclonal anti-DNA that induces experimental systemic lupus erythematosus in mice is encoded by a VH4 gene segment. Int Immunol 1995; 7:689-96.
5. Luger D, Dayan M, Zinger, H., Liu J-P, Mozes E. A peptide based on the complementary determining region 1 of a human monoclonal autoantibody ameliorates spontaneous and induced lupus manifestations in correlation with cytokine immunomodulation. J. Clin. Invest. 2004; 24:579-90.
6. Sharabi A, Haviv A, Zinger H, Dayan M, Mozes E. Amelioration of murine lupus by a peptide, based on the complementary determining region-1 of an autoantibody as compared to dexamethasone: Different effects on cytokines and apoptosis. Clin Immunol. 2006; 119:146-55.
7. Rapoport M J, Sharabi A, Aharoni D, Bloch 0, Zinger H, Dayan M, Mozes E. Amelioration of SLE-like manifestations in (NZB×NZW)F1 mice following treatment with a peptide based on the complementary determining region 1 of an autoantibody is associated with a down-regulation of apoptosis and of the pro-apoptotic factor JNK kinase. Clin Immunol. 2005; 117:262-70.
8. Sela U, Hershkovic R, Cahalon L, Lider O, Mozes E. Down-regulation of stromal cell-derived factor-1 alpha-induced T cell chemotaxis by a peptide based on the complementary-determining region 1 of an anti-DNA autoantibody via up-regulation of TGF-beta secretion. J. Immunol. 2005; 174:302-9.
9. Sela U, Mauermann N, Hershkovic R, Zinger H, Dayan M, Cahalon L, Liu J P, Mozes E, Lider O. The inhibition of autoreactive T cell functions by a peptide based on the CDR1 of an anti-DNA autoantibody is via TGF-beta-mediated suppression of LFA-1 and CD44 expression and function. J. Immunol. 2005; 175:7255-63.
10. Wakeland E K, Liu K, Graham R R, Behrens T W. Delineating the genetic basis of systemic lupus erythematosus. Immunity. 2001; 15:397-408.
11. Mozes E, Ailing D, Miller M W, Payne S M, Zinger H, Via C S Shearer G M. Genetic analysis of experimentally induced lupus in mice. Clin. Immunol. Immunopathol. 1997; 85:28-34.
12. McDevitt H O. The role of MHC class II molecules in susceptibility and resistance to autoimmunity. Curr. Opin. Immunol. 1998; 10:677-81.
13. Ridgway W M, Fasso M, Fathman C G. A new look at MHC and autoimmune disease. Science 1999; 284:749-51.
14. Bolland S, Yim Y S, Tus K, Wakeland E K, Ravetch J V. Genetic modifiers of systemic lupus erythematosus in Fc□RIIB^{-/-} mice. J. Exp. Med. 2002; 195:1167-74.
15. Rus V, Atamas S P, Shustova V, Luzina I G, Selaru F, Magder L S, Via C S. Expression of cytokine- and chemokine-related genes in peripheral blood mononuclear cells from lupus patients by cDNA array. Clinical Immunology 2002; 102:283-90.
16. Rus V, Chen H, Zernetkina V, Magder L S, Mathai S, Hochberg M C, Via CS. Gene expression profiling in peripheral blood mononuclear cells from lupus patients with active and inactive disease. Clinical Immunology 2004; 112:231-4.
17. Maas K., Chan S, Parker, Slater A, Moore J, Olsen N, Aune T M. Cutting edge: molecular portrait of human autoimmune disease. J. Immunol. 2002; 169:5-9.
18. Deng Y J, Huang Z X, Zhou C J, Wang J W, You Y., Song Z Q, Xiang M M, Zhong B Y, Hao F. Gene profiling involved in immature CD4+T lymphocyte responsible for systemic lupus erythematosus. Molecular Immunology 2006; 43:1497-1507.
19. Oelke K, Lu Q, Richardson D, Wu A, Deng C, Hanash S, Richardson B. Overexpression of CD70 and overstimulation of IgG synthesis by lupus T cells and T cells treated with DNA methylation inhibitors. Arthritis and Rheumatism 2004; 50:1850-60.
20. Xu L, Zhang L, Yi Y, Kang H-K, Datta S K. Human lupus T cells resist inactivation and escape death by up-regulating COX-2. Nature medicine 2004; 10:411-5.
21. Qing X, Putterman C. Gene expression profiling in the study of the pathogenesis of systemic lupus erythematosus. Autoimmunity Reviews 2004; 3:505-9.
22. Alexander J J, Saxena A K, Bao L, Jacob A, Haas M, Quigg R J. Prominent renal expression of a murine leukemia retrovirus in experimental systemic lupus erythematosus. J Am Soc Nephrol 2002; 13:2869-77.
23. Gentleman R C, Carey V J, Bates D M, Bolstad B, Dettling M, Dudoit S, et al. Bioconductor: open software development for computational biology and bioinformatics. Genome Biol. 2004; 5:R80.
24. Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003; 4:249-64.
25. Smyth, G. K. (2004). Linear models and empirical Bayes methods for assessing differential expression in microarray experiments. Statistical Applications in Genetics and Molecular Biology 3, No. 1, Article 3.
26. Aten J, Roos A, Claessen N, Schilder-Tol E J, Ten Berge I J M, Weening J J. Strong and selective glomerular localization of CD 134 ligand and TNF receptor-1 in proliferative lupus nephritis. J Am Soc Nephrol 2000; 11:1426-38.
27. Lwaleed B S, Bass P S. Tissue factor pathway inhibitor: structure, biology and involvement in disease. Tissue factor pathway inhibitor: structure, biology and involvement in disease. J. Pathol. 2006; 208:327-39.
28. Ertenli I, Kiraz S, Celik I, Haznedaroglu I C, Erman M, Calguneri M, Kirazli S. Changes in the concentration and distribution of tissue factor pathway inhibitor in Behcet's disease and systemic lupus erytematosus: effect on the prethrombotic state. Ann Rheum Dis 2001; 60:1149-51.
29. Foell D and Roth J. Proinflammatory S100 proteins in arthritis and autoimmune disease. Arthritis and Rheumatism. 2004; 50:3762-71.
30. Sakamoto-Hojo E T, Mello S S, Pereira E, Fachine A L, Cardoso R S, Junta C M et al. Gene expression profiles in human cells submitted to genotoxic stress. Mutation Research 2003; 544:403-413.
31. Takatsu K. Interleukin 5 and B cell differentiation. Cytokine and growth factor reviews 1998; 9:25-35.
32. Stogios P J, Downs G S; Jauhal J J S, Nandra S K, Privé G G. Sequence and structural analysis of BTB domain proteins. Genome Biology 2005; 6:R82 1-17.
33. Sun G, Liu X, Mercado P. Jenkinson S R, Kypriotou M, Feigenbaum L, Galera P, Bosselut R. The zinc finger protein cKrox directs CD4 lineage differentiation during intrathymic T cell positive selection. Nature immunology. 2005; 6:373-81.
34. Zhen Q L, Chun X, Wu T, Mackay M, Aranow C, Putterman C, Mohan C. Identification of autoantibody clusters that best predict lupus disease activity using glomerular proteome arrays. J Clin Invest 2005; 115:3428-39.
35. Godfrey W R, Fagnoni F F, Harara M A, Buck D, Engleman E E. Identification of a human OX-40 ligand, a co-stimulator of CD4+T cells with homology to tumor necrosis factor. J. Exp.Med. 1994; 180:757-62.
36. Stüber ER, Neurath M F, Calderhead D, Fell H P, Strober W. Cross-linking of OX40 ligand, a member of the TNF/NGF cytokine family, induces proliferation and differentiation in murine splenic B cells. Immunity 1995; 2:507-21.
37. Imura A, Hori T, Imada K, Ishikawa T, Tanaka Y, Maeda M et al. The human OX40/gp34 system directly mediates adhesion of activated T cells to vascular endothelial cells. J Exp Med. 1996; 183:2185-95.
38. Weinberg A D. OX40: targeted immunotherapy-implications for tempering autoimmunity and enhancing vaccines. Trends Immunol. 2002; 23:102-9.
39. Wang X, Ria M, Kelmenson P M, Eriksson P, Higgins D C, Samnegard A et al. Positional identification of TNFSF4, encoding OX40 ligand, as a gene that influences atherosclerosis susceptibility. Nature genetics. 2005; 37:365-72.
40. Mandel M, Gurevich M, Pauzner R, Kaminski N, Achiron A. Autoimmunity gene expression portrait: specific signature that intersects or differentiates between multiple sclerosis and systemic lupus erythematosus. Clin Exp Immunol 2004; 138:164-170.
41. Budman D R, Steinberg A D. Hematologic aspects of systemic lupus erythematosus: current concepts. Ann Interm Med. 1977; 86:220-9.

1. A method of treating systemic lupus erythematosus (SLE) in a subject, the method comprising upregulating in cells of the subject activity and/or expression of at least one gene selected from the group consisting of Mpo, Ltf, Lcn, Camp, Ngp, Slfn, Ctsg, Thbs1, S100a8, 1190003K14Rik, Prtn3, S100a9, and Tfpi, thereby treating SLE.

2. A method of treating systemic lupus erythematosus (SLE) in a subject, the method comprising downregulating in cells of the subject activity and/or expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20, thereby treating SLE.

3. A method of treating systemic lupus erythematosus (SLE) in a subject, the method comprising:
(a) determining a level of expression of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject, so as to identify an altered expression of said at least one gene relative to a normal control sample; and
(b) administering an anti SLE therapy to the subject according to said level of expression of said at least one gene, thereby treating SLE in the subject.

4. The method of claim 3 further comprising repeating step (a) following step (b).

5. A method of monitoring treatment against systemic lupus erythematosus (SLE) in a subject, the method comprising:
(a) administering an anti SLE therapy to the subject; and
(b) determining a level of expression of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject following said administering, thereby monitoring treatment against said SLE in the subject.

6. The method of claim 5, further comprising performing step (b) prior to step (a).

7. A method of diagnosing systemic lupus erythematosus (SLE) in a subject in need thereof, the method comprising determining a level of expression of at least one gene selected from the group consisting of Mpo, Ltf, Lcn, Camp, Ngp, Slfn, Ctsg, Thbs1, S100a8, 1190003K14Rik, Prtn3, S100a9, and Tfpi in cells of the subject, wherein an expression lower than a predetermined threshold of said at least one gene is indicative of SLE in the subject.

8. A method of diagnosing systemic lupus erythematosus (SLE) in a subject in need thereof, the method comprising determining a level of expression of at least one gene selected from the group consisting of Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20 in cells of the subject, wherein an expression higher than a predetermined threshold of said at least one gene is indicative of SLE in the subject.

9. A kit for diagnosing systemic lupus erythematosus (SLE) the kit comprising agents directed for specific detection of at least one gene selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20.

10. The method of claim 3, wherein said anti SLE therapy is selected from the group consisting of corticosteroids, an anti malarial, an NSAID, a DMARD, CellCept (mycophenolate mofetil; MMF), Orencia® (abatacept; CTLA4-Ig), Riquent™ (abetimus sodium; LJP 394), Prestara™ (praserone), Edratide (TV-4710), Actemra® (tocilizumab; atlizumab), VX-702, TRx 1, IPP-201101, ABR-215757, sphingosine-1-phosphate-1 (S1P1) agonist, HuMax-Inflam™ (MDX 018), MEDI-545 (MDX-1103/1333), RhuDex®, Deoxyspergualin, ENBREL™ (Etanercept), anti-TNF antibody, anti-interferon-alpha antibody and an anti Neutrokine-alpha protein.

11. A method of monitoring treatment in a subject having systemic lupus erythematosus (SLE) comprising:
(a) administering to the subject a peptide as set forth in SEQ ID NO:1 (Edratide™);
(b) analyzing expression of at least one gene which expression level is altered in SLE following said administering; and
(c) identifying the subject as a responder to treatment with the peptide of SEQ ID NO:1 if gene expression level have been restored to normal, thereby monitoring SLE treatment.

12. The method of claim 11, wherein said gene is selected from the group consisting of Mpo, Ltf, Lcn, Camp, Ngp, Slfn, Ctsg, Thbs1, S100a8, 1190003K14Rik, Prtn3, S100a9, Tfpi, Fzd6, Nid1, 5830484A20Rik, 5830484A20 LOC 545340, Tnfsf4, IPstpip2, Pigr, 270022B06Rik, L5Rα, A130040M12Rik, Gpr132, Cd8b1, Dhx9, Cyp11a1, Lmo7, Rnf184, Pstpip2, Hdgfrp3, Ass1 and Zbtb20.

13. The method of claim 1 wherein said cells comprise peripheral blood lymphocytes.

14. The method of claim 2, wherein said cells comprise peripheral blood lymphocytes.

15. The method of claim 3, wherein said cells comprise peripheral blood lymphocytes.

16. The method of claim 5, wherein said cells comprise peripheral blood lymphocytes.

17. The method of claim 7, wherein said cells comprise peripheral blood lymphocytes.

18. The method of claim 8, wherein said cells comprise peripheral blood lymphocytes.

19. The method of claim 5, wherein said anti SLE therapy is selected from the group consisting of corticosteroids, an anti malarial, an NSAID, a DMARD, CellCept (mycophenolate mofetil; MMF), Orencia® (abatacept; CTLA4-Ig), Riquent™ (abetimus sodium; LJP 394), Prestara™ (praserone), Edratide (TV-4710), Actemra® (tocilizumab; atlizumab), VX-702, TRX 1, IPP-201101, ABR-215757, sphingosine-1-phosphate-1 (S1P1) agonist, HuMax-Inflam™ (MDX 018), MEDI-545 (MDX-1103/1333), RhuDex®, Deoxyspergualin, ENBREL™ (Etanercept), anti-TNF antibody, anti-interferon-alpha antibody and an anti Neutrokine-alpha protein.


SEQUENCE LISTING (15 sequences)

SEQ ID NO: 1 - protein, 19 aa, Artificial sequence
  hCDR (Edratide) synthetic peptide
GYYWSWIRQPPGKGEEWIG

SEQ ID NO: 2 - DNA, 16 bp, Artificial sequence
  Single strand DNA oligonucleotide
gtgacgttgacatccg

SEQ ID NO: 3 - DNA, 16 bp, Artificial sequence
  Single strand DNA oligonucleotide
cagtaacagtccgcct

SEQ ID NO: 4 - DNA, 16 bp, Artificial sequence
  Single strand DNA oligonucleotide
agttcggtttgcatgg

SEQ ID NO: 5 - DNA, 17 bp, Artificial sequence
  Single strand DNA oligonucleotide
gtaagcaggtcgaggtg

SEQ ID NO: 6 - DNA, 19 bp, Artificial sequence
  Single strand DNA oligonucleotide
catgctattgtatgccgag

SEQ ID NO: 7 - DNA, 17 bp, Artificial sequence
  Single strand DNA oligonucleotide
cgtcacctatggtcact

SEQ ID NO: 8 - DNA, 16 bp, Artificial sequence
  Single strand DNA oligonucleotide
tcgaaatcccgtcggt

SEQ ID NO: 9 - DNA, 16 bp, Artificial sequence
  Single strand DNA oligonucleotide
gcggagtagattcggt

SEQ ID NO: 10 - DNA, 20 bp, Artificial sequence
  Single strand DNA oligonucleotide
accagtttagccaattatgt

SEQ ID NO: 11 - DNA, 16 bp, Artificial sequence
  Single strand DNA oligonucleotide
ccagcaatcacctcca

SEQ ID NO: 12 - DNA, 18 bp, Artificial sequence
  Single strand DNA oligonucleotide
ccgtctgaactggagaag

SEQ ID NO: 13 - DNA, 17 bp, Artificial sequence
  Single strand DNA oligonucleotide
ccagaagctctgctact

SEQ ID NO: 14 - DNA, 16 bp, Artificial sequence
  Single strand DNA oligonucleotide
ttcgtgtacggtggct

SEQ ID NO: 15 - DNA, 16 bp, Artificial sequence
  Single strand DNA oligonucleotide
acgataatcccgacgc